(19) United States
(12) Patent Application Publication
(10) Pub. No.: US 2026/0060743 A1
(43) Pub. Date: Mar. 5, 2026

(54) SYSTEM FOR BIOLOGICAL TISSUE VAPORIZATION IN A LIQUID MEDIUM

(71) Applicant: Boston Scientific Scimed, Inc., Maple Grove, MN (US)

(72) Inventors: Christian BALKOVEC, Cambridge (CA); Steven KINIO, Pickering (CA); Gareth DAVIES, Toronto (CA)

(21) Appl. No.: 19/307,997

(22) Filed: Aug. 22, 2025

Related U.S. Application Data
(60) Provisional application No. 63/688,749, filed on Aug. 29, 2024.

(51) Int. Cl. A61B 18/14; A61B 18/00

(52) U.S. Cl. CPC A61B 18/1477; A61B 18/1492; A61B 2018/00077; A61B 2018/00083; A61B 2018/00351; A61B 2018/00601; A61B 2018/00625; A61B 2018/00642; A61B 2018/00827; A61B 2018/00892; A61B 2018/144; A61B 2018/147

(57) ABSTRACT

An electrosurgical system to puncture biological target tissue is disclosed. The electrosurgical system includes an electrosurgical generator to generate a radiofrequency (RF) energy, and a crossing member having a distal end to be disposed within a conductive liquid medium proximate the biological target tissue. The distal end having an electrode adapted to deliver the RF energy. The electrode to apply the RF energy to generate an electrically insulative gaseous layer within the conductive liquid medium to encapsulate the electrode and to vaporize the biological target tissue from within the electrically insulative gaseous layer.

### CROSS REFERENCE TO RELATED APPLICATIONS

This application claims priority to U.S. Provisional Application No. 63/688,749 entitled “SYSTEM FOR BIOLOGICAL TISSUE VAPORIZATION IN A LIQUID MEDIUM,” filed Aug. 29, 2024, which is herein incorporated by reference in its entirety.

### TECHNICAL FIELD

The present disclosure relates to medical devices and systems for use in percutaneous or interventional procedures including surgery such as electrophysiology procedures. More specifically, this disclosure relates to electrosurgical devices, assemblies, and systems to puncture bodily tissues such as the atrial septum with an electrode.

### BACKGROUND

Catheters are often used to provide general access into a patient's body using minimally invasive techniques. In some examples, a catheter can be used to create a channel through a region of the body. One such example is a transseptal puncture in a cardiac procedure. The left atrium is a difficult cardiac chamber to access percutaneously. Although the left atrium can be reached via the left ventricle and mitral valve, the catheter is manipulated through two U-turns, which can be cumbersome. the transseptal puncture is a technique of creating a small surgical passage through the atrial septum, or wall in the heart between the left and right atrium, through which a catheter can be fed. The atrial septum is punctured and dilated via tools to create the passage. The transseptal puncture permits a direct route to the left atrium via the atrial septum and systematic venous system. Increasing larger and complex medical devices can be passed into the left atrium. Historically, the technique was used exceptionally for mitral valvuloplasty and ablation in the left heart. Today, the increased interest in catheter ablation and its application in many other procedures has meant the transseptal puncture is a routine technique for interventional cardiologists and cardiac electrophysiologists.

Transseptal punctures can be performed with the aid of crossing devices having electrodes energized with a suitable power source such as an electrically coupled power generator in a manner like other electrosurgical devices. Typical electrosurgical devices apply an electrical potential difference or a voltage difference between an active electrode and a return electrode on a patient's grounded body in a monopolar arrangement or between an active electrode and a return electrode on the device in bipolar arrangement to deliver electrical energy to the area where tissue is to be affected. Electrosurgical devices pass electrical energy through tissue between the electrodes to puncture tissue with plasma formed on the energized electrode. Tissue that contacts the plasma experiences a rapid vaporization of cellular fluid to produce a puncturing effect. Electrical energy can be applied to the electrodes either as a train of high frequency pulses or as a continuous signal typically in the radiofrequency (RF) range to perform the puncturing techniques.

### SUMMARY

In Example 1, an electrosurgical system configured to puncture biological target tissue, the electrosurgical system comprising: an electrosurgical generator configured to generate a radiofrequency (RF) energy; and a crossing member having a distal end adapted to be disposed within a conductive liquid medium proximate the biological target tissue, the distal end having an electrode adapted to deliver the RF energy, wherein the electrode is configured to apply the RF energy to generate an electrically insulative gaseous layer within the conductive liquid medium to encapsulate the electrode and to vaporize the biological target tissue from within the electrically insulative gaseous layer.

In Example 2, the electrosurgical system of Example 1, wherein the electrosurgical generator is configured to drive a current and voltage within a selected range against a plurality of impedances.

In Example 3, the electrosurgical system of any of Examples 1 and 2, wherein the electrode includes a surface area in a range between 1.2 square millimeters and 3.5 square millimeters and the electrosurgical unit provides 270 volts to the electrode.

In Example 4, the electrosurgical system of any of Examples 1-3, wherein the electrode is an exposed section of an electrical conductor on the crossing member.

In Example 5, the electrosurgical system of Example 4, wherein the crossing member includes an electrically insulated shaft.

In Example 6, the electrosurgical system of any of Examples 1-5, wherein the electrode includes a cylindrical shape.

In Example 7, the electrosurgical system of Example 6, wherein the electrode includes circular edges.

In Example 8, the electrosurgical system of any of Examples 1-7, wherein the electrically insulative gaseous layer includes a coalesced bubble adhered to the electrode.

In Example 9, the electrosurgical system of Example 8, wherein the coalesced bubble is formed from a plurality of bubbles.

In Example 10, the electrosurgical system of any of Examples 1-9, wherein the electrosurgical generator includes a feedback controller having a voltage-feedback control.

In Example 11, the electrosurgical system of Example 10, wherein the feedback controller further includes a selectable current-feedback control.

In Example 12, the electrosurgical system of Example 11, wherein the current-feedback control is selected during a cutting phase.

In Example 13, the electrosurgical system of Example 12, wherein the voltage-feedback control is selected when the cutting phase is complete.

In Example 14, the electrosurgical system of any of Examples 1-13, wherein the crossing member is included in a crossing system having a sheath and a dilator.

In Example 15, the electrosurgical system of any of Examples 1-14, wherein the crossing member is a transseptal guidewire.

In Example 16, an electrosurgical system configured to puncture biological target tissue, the electrosurgical system comprising: an electrosurgical generator configured to generate a radiofrequency (RF) energy; and a crossing member having a distal end adapted to be disposed within a conductive liquid medium proximate the biological target tissue, the distal end having an electrode adapted to deliver the RF energy, wherein the electrode is configured to apply the RF energy to generate an electrically insulative gaseous layer within the conductive liquid medium to encapsulate the electrode and to vaporize the biological target tissue from within the electrically insulative gaseous layer.

In Example 17, the electrosurgical system of Example 16, wherein the electrosurgical generator is configured to drive a current and voltage within a selected range against a plurality of impedances.

In Example 18, the electrosurgical system of Example 16, wherein the electrode includes a surface area in a range between 1.2 square millimeters and 3.5 square millimeters and the electrosurgical unit provides 270 volts to the electrode.

In Example 19, the electrosurgical system of Example 16, wherein the electrode is an exposed section of an electrical conductor on the crossing member.

In Example 20, the electrosurgical system of Example 19, wherein the crossing member includes an electrically insulated shaft.

In Example 21, the electrosurgical system of Example 16, wherein the electrode includes a cylindrical shape.

In Example 22, the electrosurgical system of Example 21, wherein the electrode includes circular edges.

In Example 23, the electrosurgical system of Example 16, wherein the electrically insulative gaseous layer includes a coalesced bubble adhered to the electrode.

In Example 24, the electrosurgical system of Example 23, wherein the coalesced bubble is formed from a plurality of bubbles.

In Example 25, the electrosurgical system of Example 16, wherein the electrosurgical generator includes a feedback controller having a voltage-feedback control.

In Example 26, the electrosurgical system of Example 25, wherein the feedback controller further includes a selectable current-feedback control.

In Example 27, the electrosurgical system of Example 26, wherein the current-feedback control is selected during a cutting phase.

In Example 28, the electrosurgical system of Example 27, wherein the voltage-feedback control is selected when the cutting phase is complete.

In Example 29, the electrosurgical system of Example 16, wherein the crossing member is included in a crossing system having a sheath and a dilator.

In Example 30, the electrosurgical system of Example 16, wherein the crossing member is a transseptal guidewire.

In Example 31, a method of puncturing a biological target tissue with an electrode coupled to a source of radiofrequency (RF) energy, the method comprising: while the electrode is within the conductive liquid medium, encapsulating the electrode with an electrically insulative gaseous layer; and vaporizing the biological target tissue via the RF energy applied to the electrode encapsulated by the electrically insulative gaseous layer.

In Example 32, the method of Example 31, wherein the electrically insulative gaseous layer includes coalesced bubbles adhered to the electrode.

In Example 33, the method of Example 31, wherein the electrode is disposed within a sheath, and the conductive liquid medium is disposed within the sheath.

In Example 34, an electrosurgical system of configured to puncture biological target tissue, the electrosurgical system comprising: an electrosurgical generator configured to generate a radiofrequency (RF) energy; and a crossing member assembly having a delivery device defining a lumen and crossing member disposed within the lumen, the lumen configured to include a conductive liquid medium, the crossing member having a distal end adapted to be disposed within the conductive liquid medium, the distal end having an electrode adapted to deliver the RF energy, wherein the electrode is configured to apply the RF energy to generate an electrically insulative gaseous layer within the lumen and the conductive liquid medium to encapsulate the electrode and to extend from the delivery device to vaporize the biological target tissue from within the electrically insulative gaseous layer

In Example 35, the electrosurgical system of Example 34, wherein the delivery device is a sheath.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1A is a schematic diagram illustrating an example electrosurgical system for treating a patient, such as a heart or the vasculature of a patient, including an electrosurgical generator and a transseptal crossing system.

FIG. 1B is a cutaway view of a heart including an atrial septum and the transseptal crossing system of FIG. 1A.

FIG. 1C is a side view of a distal end of the transseptal crossing system of FIG. 1A.

FIG. 2 is a block diagram illustrating an example process of the electrosurgical system of FIG. 1A.

FIG. 3 is block diagram illustrating an example process that is implemented in the process of FIG. 2.

FIGS. 4A-4D are schematic diagrams illustrating an example crossing member for use in the example electrosurgical system of FIG. 1A in various phases of the method of FIG. 3.

FIGS. 5A-5C are schematic diagrams illustrating the example crossing member of FIGS. 4A-4D in an embodiment of the method of FIG. 3.

FIGS. 6A-6B are schematic diagrams illustrating the example crossing member of FIGS. 4A-4D in another embodiment of the method of FIG. 3.

FIGS. 7A and 7B are schematic diagrams illustrating a crossing member likely unsuitable for the application of the method of FIG. 3.

FIGS. 8A and 8B are schematic diagrams illustrating another crossing member likely unsuitable for the application of the method of FIG. 3.

FIGS. 9A and 9B are schematic diagrams illustrating an example crossing member assembly in an embodiment of the method of FIG. 3.

FIG. 10 is schematic diagram illustrating the example crossing member assembly of FIG. 9B in the embodiment of the method of FIG. 3.

FIG. 11 is a block diagram illustrating an example electrosurgical generator of the example, electrosurgical system of FIG. 1A.

FIG. 12 is a block diagram illustrating an example feedback loop controller of the example electrosurgical generator of FIG. 11.

FIG. 13 is a flow chart illustrating an example process of the example electrosurgical generator of FIG. 11 implementing the example feedback loop controller of FIG. 12.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

The vaporization of biological target tissue in a conductive liquid medium, such as blood, can be performed by delivering radiofrequency (RF) energy through a conductive crossing member. Typically, the conductive crossing member is electrically insulated with the exception of a small distal portion, formed as an electrode, that is intended to deliver the RF energy to the tissue. To achieve tissue vaporization, the selected tissue is rapidly heated. If heating is too slow, the tissue is desiccated rather than vaporized. Rapid heating is achieved through high current density at the electrode-tissue interface, meaning the electrode is either relatively small or the energy delivered is relatively high.

Achieving high current density in electrodes for tissue vaporization can be a challenge. High amounts of heat are generated during this process, meaning that the dielectric material surrounding the conductive crossing member and proximate to the conductive electrode can withstand the heat. Further, the crossing member presents a limit on the achievable current density as all electrically insulative materials eventually break down at a given thermal level. There is also a desire to have a large enough conductive surface of the electrode such that physical trauma to the tissue environment is avoided (i.e. if the fixed surface area of the conductive electrode is too small, it could form a sharp interface that can cause tissue trauma). Accordingly, the electrode is configured to be atraumatic.

When the electrode is operating in a conductive liquid environment such as the bloodstream, any surface area of the electrode in contact with the surrounding liquid environment instead of the target tissue represents current that is being deposited into the bloodstream and not being delivered to the tissue, which decreases the efficiency of the tissue vaporization process. If the energy delivered in the effort to achieve high current density at the electrode-tissue interface is excessively high, however, inadvertent coagulation of blood can occur via the regions of the electrode directly in contact with the liquid environment. Conversely, if the electrode size is minimized excessively, fulguration can occur with all media in contact with the electrode and eschar or thrombotic material is formed. In either of these scenarios, the formation of thrombotic material in the bloodstream can lead to embolization and subsequent deleterious health effects to the patient.

Examples of the devices, systems, and methods of the disclosure are presented in the context of a transeptal puncture for illustration purposes. Those skilled in the art will recognize other applicable contexts. For example, electrosurgical systems of the disclosure can be employed to puncture a pericardium layer of a patient for epicardial access or to remove accumulation of atheromatous material on the inner walls of vascular lumens.

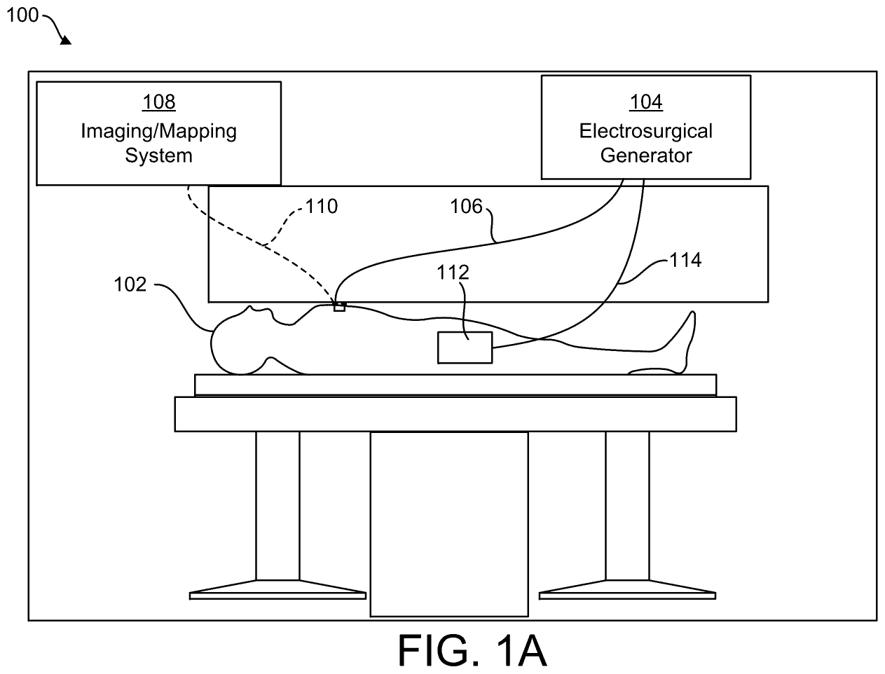

FIG. 1A illustrates an electrosurgical system 100 for treating a patient 102. The system 100 includes an electrosurgical generator 104 with a transseptal crossing system 106 and an imaging/mapping system 108 for tracking the crossing system 106 in the patient 102. The imaging/mapping system 108 can use an external fluoroscopy system (not shown) and/or a mapping catheter 110 (shown in phantom) (such as, for example, the OPAL HDx™ mapping system from the Boston Scientific Corporation).

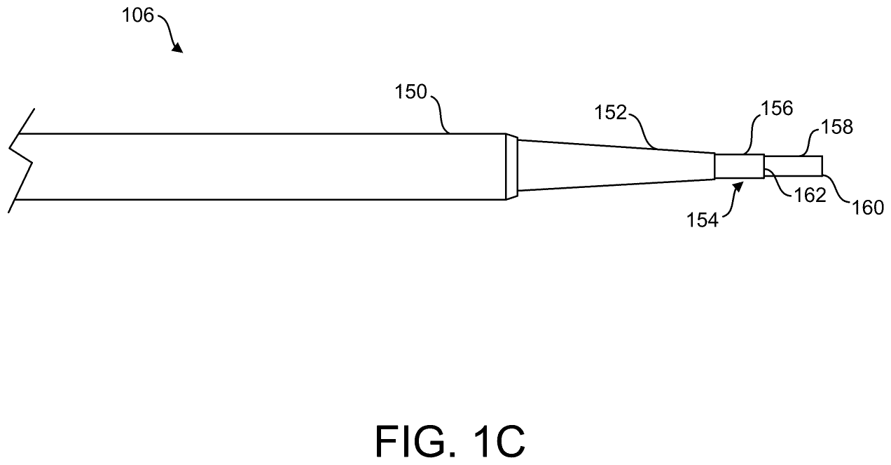

The electrosurgical generator 104 is configured to provide energy, such as radiofrequency (RF) electrical energy, to the crossing system 106. Typically, the conductive crossing system 106 is electrically insulated except for a small distal portion, formed as a vaporizing electrode (shown in FIG. 3), that is intended to deliver the RF energy to the target tissue. To achieve tissue vaporization, the target tissue is rapidly heated. If heating is too slow, the tissue is desiccated rather than vaporized. Rapid heating is achieved through high current density at the electrode-tissue interface, meaning the electrode is either relatively small and/or the energy delivered is relatively high.

The delivery of energy to the target tissue can also heat the crossing system 106 itself. Thus, the electrical insulation around the crossing system 106 (especially near the electrode) should be able to withstand such heat without breaking down. While typically the insulation has been made from per- and polyfluoroalkyl substances (PFAS) (e.g., polytetrafluoroethylene (PTFE)), which provide an advantage over other materials in heat performance, so the crossing system 106 is designed to reduce the heat generation within itself while still providing adequate heat generation in the target tissue.

In the illustrated embodiment of FIG. 1, the crossing system 106 is monopolar and includes a single vaporizing electrode (i.e., an active electrode), so the system 100 also includes a patch electrode 112 (i.e., an indifferent or dispersive electrode). The patch electrode 112 has a large surface area to lower current density, so the patch electrode 112 is typically located on the back, buttocks, or upper leg of the patient 102, although there may be other suitable locations as well. The patch electrode 112 returns the RF electrical energy to the generator 104 through the lead 114. In some embodiments, the RF energy for a monopolar puncture function is provided by the electrosurgical generator 104 at a selected voltage and a continuous current (100% on, or 100% duty cycle). For example, if a power setting of 50 watts (W) is used for puncturing (which can mean that the instantaneous power is higher than 50 W), the voltage can range from approximately 164 volts (V) to 400 V root mean square (RMS).

In addition, the electrosurgical generator 104 can include a plurality of functions and provide programmed and custom settings via an interface (not shown). For example, the electrosurgical generator 104 provides RF energy to the crossing system 106 as an alternating current having a frequency in the range of 100 kilohertz (kHz) to 10 megahertz (MHz). Such puncturing RF energy can be applied in the form of a continuous waveform signal or in bursts of a waveform signal. In the latter case, the individual bursts of the waveform signal can have a duration of 300 milliseconds (ms) with a rest interval between pulses of 700 ms, although other durations of bursts and intervals can be used. In some embodiments, the waveform signals themselves can be sinusoidal or square waves that are bi-phasic. Furthermore, the electrosurgical generator 104 can be couplable to other electrosurgical tools and the electrosurgical generator 104 can receive signals (e.g., from the crossing system 106) to monitor the patient 102.

The components and configuration of electrosurgical system 100 allow for target tissue to be vaporized. In some embodiments, the tissue vaporization allows the crossing system 106 to puncture through the atrial septum for treatment of the left side of the heart of the patient 102. While examples of the devices, systems, and methods of the present disclosure are presented in the context of a transeptal puncture, a person having ordinary skill in the art will recognize other applicable contexts. For example, the electrosurgical systems of the present disclosure can be employed to puncture a pericardium layer of a patient for epicardial access and/or to remove accumulation of atheromatous material on the inner walls of vascular lumens.

FIG. 1B illustrates a heart 130 of the patient 102 (shown in FIG. 1A) with selected portions cut away. The crossing system 106 extends through the inferior vena cava 132 from a surgical entry site (not shown) that is distal to the heart 130. The distal end of the crossing system 106 is positioned in the right atrium 134 and is in contact with the atrial septum 136. In the illustrated embodiment, the tip of the crossing system 106 is positioned at the fossa ovalis because this region of the atrial septum 136 is relatively safe and easy to puncture. Once the atrial septum 136 is crossed, the physician will have access to the left atrium 138 (e.g., for treatment thereof).

In some use cases, such as a septal crossing, the electrode will be surrounded by a conductive liquid medium, such as blood, that is near the target tissue. When the crossing system 106 is operating in such an environment, any surface of the electrode that is in contact with the conductive liquid (instead of with the target tissue) provides a shunt path for the electrical current. These alternative electrical pathways do not help vaporize the target tissue, so they decrease the efficiency of the tissue vaporization process. Furthermore, these pathways can cause the blood to locally coagulate and form thrombotic material that can lead to embolization and subsequent deleterious health effects for the patient 102 (shown in FIG. 1A). Therefore, components and methods to prevent such occurrences are discussed in the present disclosure.

FIG. 1C illustrates a distal end of the crossing system 106. The crossing system 106 includes a sheath 150, a dilator 152, and a crossing member or device 154. The crossing device 154 is an elongated tissue vaporization device that can have the form of, for example, a wire, a needle, forceps, scalpels, or other devices that puncture and/or cut tissue. In some embodiments, a wire is a solid, stiff but elastically deformable member with a generally straight and/or helical configuration. In some embodiments, a needle is a hollow, flexible member with a generally straight configuration through which fluid can be pumped. The fluid can exit near an electrode that is positioned at the distal end of the needle, and the electrode can be connected to the electrosurgical generator 104 with a conductor since the flexible member can be made from an electrically insulating material. In some embodiments, forceps are a dual-levered instrument capable of grasping and/or holding tissue or other objects between their distal ends. In some embodiments, a scalpel is a bladed instrument with a sharpened edge capable of cutting tissue or other objects.

In some embodiments, the crossing system 106 has an overall length between about 55 centimeters (cm) and 300 cm. The sheath 150 is an elongate member with a central lumen (not shown), in which the dilator 152 and the crossing device 154 are slidably positioned. The central lumen diameter is similar to the outer diameter of the majority of the dilator 152 (except for the distal tip), and the sheath 150 is tapered at the distal end to make the transition between the sheath 150 and the dilator 152 smoother. In addition, the sheath 150 can be a steerable sheath and/or have a fixed or adjustable curve at the distal end for positioning of the dilator 152 and the crossing device 154. In some embodiments, the sheath 150 and the dilator 152 are generally similar to those of a VersaCross™ Access Solution from Boston Scientific.

In the illustrated embodiment, the dilator 152 is an elongate member with a central lumen (not shown), in which the crossing device 154 is slidably positioned. The central lumen diameter is similar to the outer diameter of the crossing device 154, and the dilator 152 is tapered at the distal end to make the transition between the dilator 152 and the crossing device 154 smoother. In addition, the dilator 152 can have a fixed or adjustable curve at the distal end for positioning of the crossing device 154 against the atrial septum 136 (shown in FIG. 1B).

In the illustrated embodiment, the crossing device 154 is an elongate member with a central electrical conductor (not shown) (e.g., comprising stainless steel, nitinol, platinum, gold, or combinations thereof) that is surrounded by an electrically insulative layer 156 (e.g., comprising parylene, polyimide, polyethylene terephthalate (PET), polyurethane, silicone ((R 2 SiO)ₓ), PTFE heat shrink, or combinations thereof. In some embodiments, a diameter of the crossing device 154 is between about 0.50 millimeters (mm) and about 1.0 mm. An electrode 158 forms the distal end of the crossing device 154, and the electrode 158 is electrically connected to the electrosurgical generator 104 (shown in FIG. 1A) via the central electrical conductor. The electrode 158 is an electrical conductor comprising, for example, platinum, gold, stainless steel, or nitinol. In some embodiments, the electrode 158 is an exposed section of the central electrical conductor, and in other embodiments, the electrode 158 is connected to the distal end of the central electrical conductor and has the same diameter as the insulative layer 156. The electrode 158 has a cylindrical shape with a flat distal end having a distal circular edge 160 and a proximal circular edge 160 where the proximal end of the electrode 158 and the distal end of the insulative layer 156 are coterminous. In some embodiments, the electrode 158 is atraumatic (i.e., not sharp) and has distinct circular edges 160, 162 at the distal and proximal ends, respectively. In other embodiments, however, the crossing device has a distal domed shape.

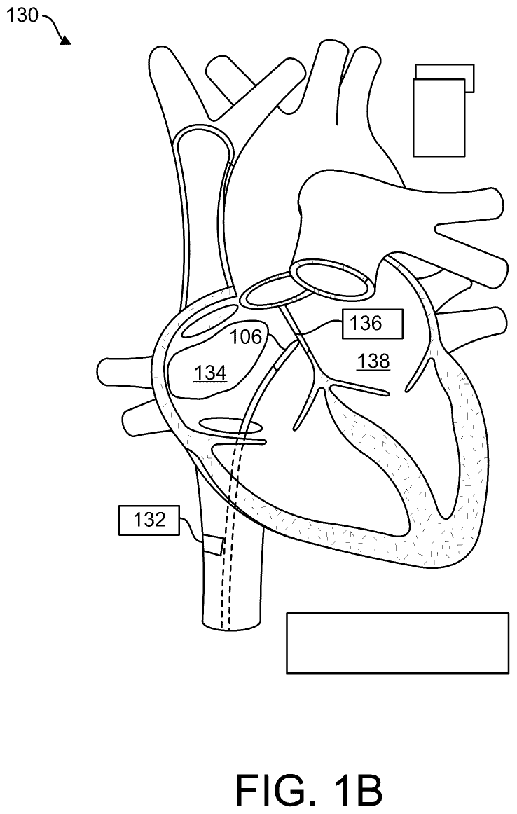

FIG. 2 illustrates an example method 200 of an anticipated use of the system 100. The transseptal crossing system 106 is coupled to the RF generator 104. If the transseptal crossing system 106 is to be configured in a monopolar mode, the patch electrode 112 is coupled to the patient 102. The RF generator 104 can be set to a puncture mode, such as an energy output of approximately 50 watts. In some examples, femoral access is obtained via a conventional percutaneous needle, and the crossing device 154 is inserted into the vasculature and advanced to the superior vena cava at 202. The distal tips of the sheath 150 and dilator 152 are advanced over the proximal portion of the crossing device 154 as a guidewire and advanced over the crossing device 154 to the superior vena cava at 204. Under visualization, the distal tip of dilator 152 is moved from the superior vena cava to the right atrial septum and then to the fossa ovalis of the heart. The location of the crossing system 106 is confirmed at the fossa ovalis at 206. For example, the distal tip of the dilator 152 is confirmed at the fossa ovalis at 206, such as via visualization, and the electrode 158 of the transseptal crossing device 154 is advanced from the distal tip of the crossing device 154.

The crossing device 154 is energized to puncture the fossa ovalis at 208. In embodiments, the energy delivered to the crossing device 154 forms an electrically insulative gaseous layer on the surface of the electrode 158 and is formed on and is maintained on the portions of the electrode in contact with a liquid medium, such as blood within the heart chamber. In one example, the exposed electrode 158 of the crossing device 154 is extended a few millimeters from the distal tip of the dilator 152 to tent the heart tissue, and the crossing device 154 can be locked in position with respect to the sheath 150 and dilator 152. Forward pressure is applied to the crossing system 106 and the crossing device 154 is actuated to apply the RF energy to the electrode 158 and puncture the fossa ovalis at 208. The electrically insulative gaseous layer formed on the electrode directs the RF energy to the biological tissue, such as the atrial septum, rather than into the current shunting conductive liquid medium such as the blood pool. The RF energy punctures the fossa ovalis and creates an aperture in the fossa ovalis.

The crossing device 154 operated as a guidewire can remain at the puncture site for additional functions. The crossing device 154 is unlocked from the delivery sheath 150 and dilator 152, and the crossing device 154 is extended through the aperture in the fossa ovalis at 210. In general, the crossing device 154 is extended longitudinally for several millimeters prior to the distal portion of the crossing device 154 curving to assume a J-tip or pigtail shape and deflecting away from the atrial septum. The crossing device 154 can be advanced into the left atrium of the heart through the aperture and anchored at 212. The distal portion of the dilator 152 is advanced into the puncture site to expand the aperture 214. The sheath 150 can be retracted from the patient over the crossing device 154 at 216, and the crossing device 154 can provide support to the installation of tubular members or other catheters and for advancing other devices within the heart through the enlarged or expanded aperture at 218.

FIG. 3 illustrates a method 300 featured in 208 of method 200 of FIG. 2 for puncturing biological target tissue, such as a fossa ovalis or other tissue. The electrode, such as electrode 158, is positioned within a conductive liquid medium, such as blood, and proximate the biological target tissue at 302. While the electrode is within the conductive liquid medium, the electrode is energized and encapsulated with an electrically insulative gaseous layer at 304. In an embodiment, the electrode is in contact with the electrically conductive liquid medium. RF energy is delivered to the electrode to heat the electrode enough to form the gaseous layer from the electrically conductive liquid medium in contact with the electrode. The biological target tissue is vaporized via the RF energy applied to the electrode encapsulated by the electrically insulative gaseous layer at 306.

In the example, the effective surface area of the electrode against the conductive liquid and tissue dynamically changes size in response to the delivery of RF energy. For example, the surface area of the electrode against the conductive liquid and tissue is not initially so small that RF energy delivery through the electrode results in fulguration of any media in its path or thermal destruction of its surrounding insulation, but also not so large that the tissue is merely desiccated. Rather, RF energy delivery cause heating of all surrounding media followed by a decrease in effective electrode surface area so that generally just the target tissue is vaporized.

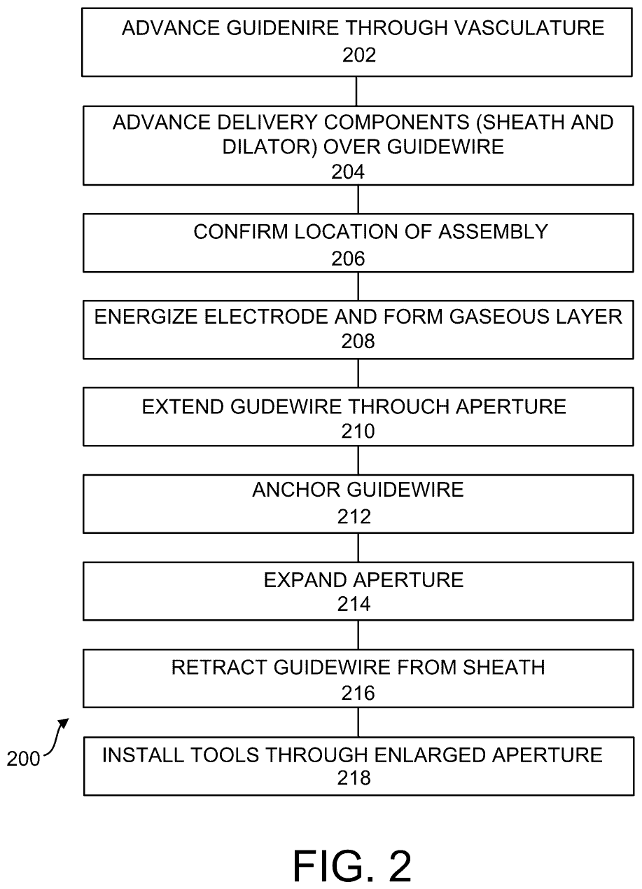

FIGS. 4A-4D illustrates features of method 300 as applied to crossing member 400. In the example, crossing member 400 can correspond with transseptal crossing device 154 of FIG. 1. FIG. 4A illustrates the crossing member 400 disposed in a conductive liquid medium 402, such as a blood stream or blood pool in a chamber of a heart, prior to the application of RF energy to the crossing member 400. Crossing member 400 includes a shaft 404 having a distal portion 406 defining a distal tip 408. The shaft 404 includes elongated conductive mandrel 410 having an electrically insulative or dielectric cover member 412 disposed on the mandrel 410. In the illustrated example, the electrically insulative cover member 412 extends to a cover member distal end 414, which is proximal to the distal tip 408. The crossing member 400 includes an exposed electrically conductive portion defining the distal electrode 420, or puncture electrode 420, distal to the cover member distal end 414. The electrode 420 is electrically coupled to the mandrel 410, and the mandrel delivers the RF energy from the proximal portion of the crossing member 400 to the electrode 420. The puncture electrode 420 includes an exposed conductive surface, which in the illustrated example includes a longitudinal surface 422 and an end surface 424 to form an edge 426 at the distal tip 408. Other configurations are possible, such as domed-shaped electrode. The surface area of the puncture electrode 420 includes the total area of the exposed conductor. In the example, the electrode 420 is a fixed size electrode such that the total surface area is not manipulable or changeable by a user.

As RF energy is delivered to the conductive electrode 420 via an energy generator, such as electrosurgical generator 104, the conductive liquid 402 surrounding the electrode 420 is heated, causing the formation of bubbles 428 of an electrically insulative gas on the surfaces 422, 424 of the electrode 420. FIG. 4B illustrates a schematic view of the crossing member 400 in the liquid 402 as the bubbles 428 begin to form when energy is initially applied to the electrode 420. In one example, the bubbles 428 are relatively small and can form on the electrode 420 proximate the cover member distal end 414 and longitudinal surface 422 interface or near the longitudinal surface 422 and end surface 424 edge 426. FIG. 4C illustrates a schematic view of the crossing member 400 in the liquid 402 as bubbles 428 continue to form when RF energy remains applied to the electrode 420. As RF energy remains applied to the electrode 420, the bubbles 428 tend to grow and lengthen, and smaller bubbles combine to form relatively larger bubbles. Relatively gradually, bubbles begin to form on more portions of the electrode surfaces 422, 424. As more bubbles 428 form on the electrode 420 from the applied RF energy, the bubbles 428 remain adhered to the surface and coalesce together to form a larger bubble encapsulating the electrode 420 as an insulative gaseous layer around the electrode 420. FIG. 4D illustrates that when subjected to heating from the applied RF energy, eventually the bubbles 428 will coalesce and encapsulate the exposed electrode 420 with an electrically insulative gaseous layer 430 disposed between the conductive electrode 420 and the conductive liquid medium 402. In the illustrated example, the plurality of bubbles form together into a single bubble as the insulative gaseous layer 430. The insulative gaseous layer 430 encapsulating the electrode 420 does not permit current in the electrode 420 from the applied RF energy to shunt into the conductive liquid medium 402.

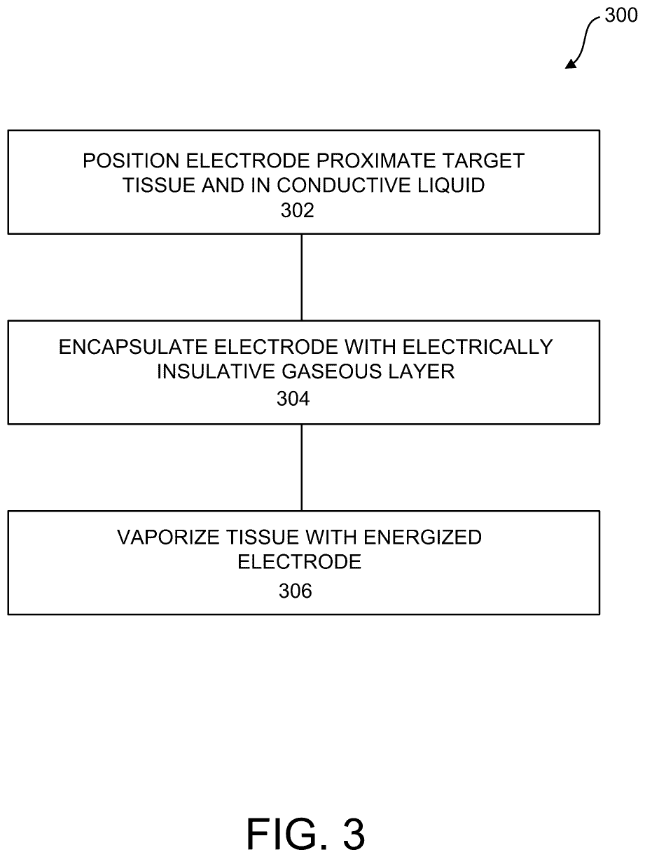

FIGS. 5A-5C illustrate a first embodiment of the crossing member 400 having the energized electrode 420 with the electrically insulative gaseous layer 430 in the liquid medium 402 applied to puncture target tissue 440. FIG. 5A illustrates the energized electrode 420 encapsulated with the insulative gaseous layer 430, as in FIG. 4D, approaching the target tissue 440. The electrode 420 is subjected to energy delivery and a bubble 430 is fully formed around the electrode 420 electrically insulating the electrode from the liquid medium 402. FIG. 5B illustrates the energized electrode 420 encapsulated with the insulative gaseous layer 430 more proximate the target tissue 440 than in FIG. 5A. As the electrode 420 is moved closer to the target tissue 440, the bubble 430 is displaced by the tissue. In the illustrated example, the distal tip 408 approaches the target tissue 440 and the gaseous layer 430 becomes thinner between the distal tip 408 and the target tissue as the gaseous layer 430 is displaced. Eventually, the gap between the energized electrode 420 and the target tissue 440 reaches a distance such that an arc discharge 446 occurs and causes a small vaporization of the target tissue 440. The insulative gaseous layer 430 is thinned enough to cause a dielectric breakdown. In this embodiment, the target tissue 440 is vaporized before the electrode 420 directly contacts the target tissue 440. FIG. 5C illustrates the energized electrode 420 next to a puncture 450 in the target tissue 440 after arc discharge in FIG. 5B. The puncture 450 created via tissue vaporization from arc discharge and dielectric breakdown of the gaseous layer 430 is relatively small, and often smaller than the outer diameter of the electrode 420. In some instances, the puncture includes a diameter of a single cell of tissue. This permits the electrode 420 to be configured in an atraumatic shape to other tissues from unintended punctures as well as reduces collateral damage to the surrounding tissue.

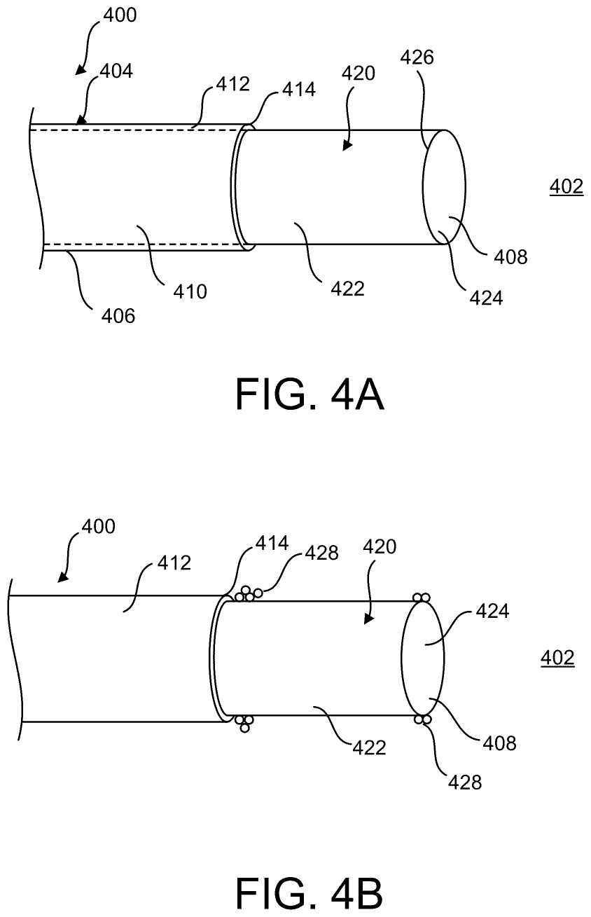

FIGS. 6A and 6B illustrate a second embodiment of the crossing member 400 applied to puncture target tissue 440. FIG. 6A illustrates the conductive electrode 420 is already in contact with the target tissue 440 and surrounded by the liquid medium 402 prior to energy delivery. As energy is delivered to the conductive electrode 420, the liquid 402 surrounding the electrode is heated, causing the formation of bubbles on the surfaces of the electrode 420, while bubbles are prevented from forming on the portions of the electrode 420 in contact with tissue 440, such as the end surface 424. As more bubbles form on the electrode 420, the bubbles remain adhered to the electrode surface and coalesce together to form the insulative gaseous layer 430 around the electrode portions in contact with the liquid environment 402. The electrically insulative gaseous layer between the electrode 420 and the liquid medium 402 prevents electrical current from being deposited into the surrounding liquid 402, allowing more current to be delivered into the tissue 440, resulting in tissue vaporization that is more efficient, requires less overall electrical current due to the reduction of electrical current being shunted into the surrounding fluid, and reduces the risk of thrombus formation. FIG. 6B illustrates the crossing member after a puncture 448 is made in the tissue via tissue vaporization. Following tissue vaporization, the gaseous layer 430 remains formed around and encapsulating the entire electrode 420, acting as an insulative layer that mitigates thrombus formation if RF energy is still being delivered to the electrode 420.

To allow for adequate adhesion of the bubbles to each other and the electrode surface for the formation of an insulative gaseous layer to encapsulate the electrode, the size of the electrode is selected relative to the energy received from the generator. For example, an electrode surface too small relative or too large relative to the amount of energy received from the generator will not properly form an insulative gaseous layer to encapsulate the electrode for a crossing procedure.

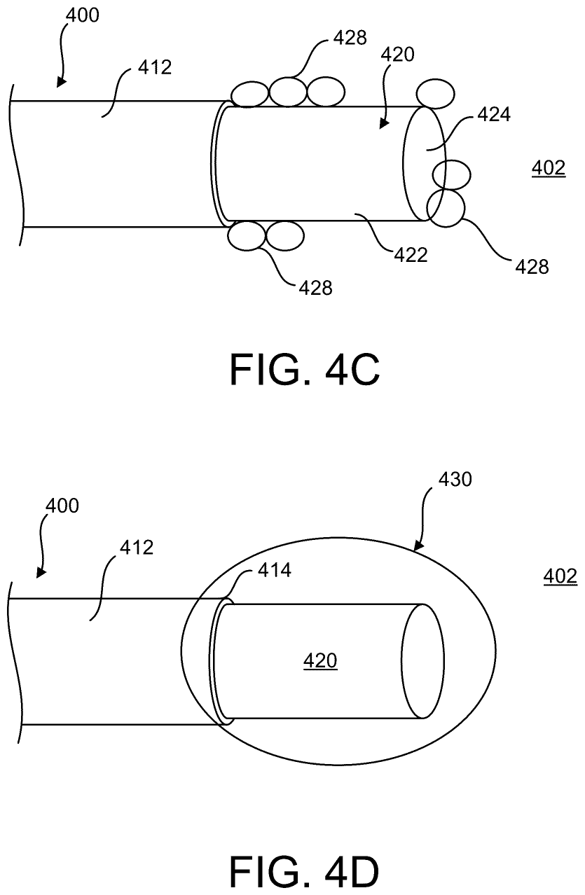

FIGS. 7A and 7B illustrate the effects of an electrode surface too small relative to an amount of energy received from a generator with example crossing member 700. Crossing member 700 including a shaft 704 having a distal portion 706 defining a distal tip 708. The shaft 704 includes an electrically insulative cover member 712 extending to the distal tip 708 such that the electrode 720 is just an exposed distal surface 724 of an electrically conductive material. If the electrode is too small relative to the energy received, a bubble 728 will form very rapidly but will be unstable on the conductive electrode surface 724, releasing from the surface and negating the benefits of the effective increased current density that results from bubble formation. FIG. 7A illustrates the bubble 728 forming rabidly from energy applied to the electrode 720, which results in a momentarily electrically insulative gaseous layer between the electrode 720 and the conductive liquid medium 702. FIG. 7B illustrates that the bubble 728 is quickly ejected from the electrode 720, and the electrode 720 is left in contact with the conductive liquid medium 702.

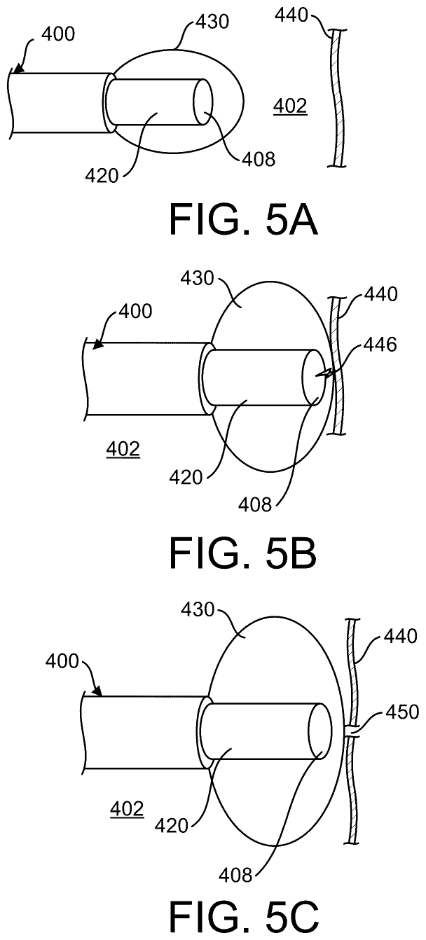

FIGS. 8A and 8B illustrate the effects of an overly large electrode surface relative to an amount of energy received from a generator with example crossing member 800. Crossing member 800 includes a shaft 804 having a distal portion 806 defining a distal tip 808. The shaft 804 includes an electrically insulative cover member 812 extending distally to a cover member distal end 814, which is proximal to the distal tip 808. A conductive electrode 820 has a relatively large exposed conductive surface, such as a relatively long longitudinal surface 822. If the electrode 820 has too large of an expose surface relative to the energy received, a cohesive bubble surrounding the electrode 840 will never form due to excessive current being shunted into the surrounding liquid medium 802. FIG. 8A illustrates bubbles 828 beginning to form on the surface of the electrode 820 as the crossing member 800 receives energy. FIG. 8B illustrates bubbles 828 are unable to coalesce to encapsulate the entire electrode 820 with an insulative gaseous layer, and the bubbles 828 detach from the electrode surface leaving portions of the electrode 820 in contact with liquid medium 802.

For a voltage-controlled system delivering 270V to the electrode, the surface area of the electrode between 1.2 mm² and 3.5 mm² provides adequate bubble formation and stability on the surface of the electrode. Electrode length also serves as a constraining factor under a given amount of energy delivery. If the length is too excessive for a given surface area, bubbles will detach from the preferential locations of formation prior to coalescing along the entire area of the electrode. Conversely, given the increased proximity of forming bubbles on a shorter electrode, they will coalesce much more readily prior to detachment and form a stable configuration surrounding the entire electrode.

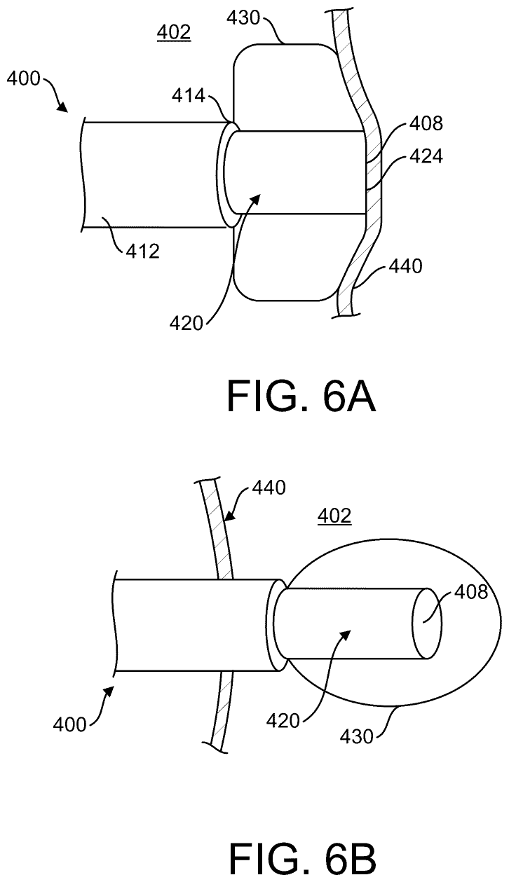

FIGS. 9A and 9B illustrate a crossing member assembly 901 having crossing member 900 inside of a delivery device 902, which in the example is configured as a tubular enclosure member. The crossing member 900 includes a shaft 904 defining a lumen 944 and having a distal portion 906 defining a distal tip 908. The shaft 904 includes an electrically insulative cover member 912 extending distally to a cover member distal end 914, which is proximal to the distal tip 908 exposing a conductive electrode 920. FIGS. 9A and 9B having a portion of the shaft 904 cutaway to illustrate the crossing member 900 within the lumen 944. FIG. 9A illustrates the assembly 901 and crossing member 900 prior to the application of RF energy to the electrode 920. The delivery device 902 is configured as a tubular enclosure member having a sidewall 940 with a distal end 942. The sidewall 940 defines a lumen 944.

In the illustrated example, the crossing member 900 is retracted into the delivery device 902 such that the distal tip 908 of the crossing member is proximal of the distal end 942 of the sidewall 940. In the illustrated example, the crossing member assembly 901 is deployed at the puncture site, so the assembly 901 is in a conductive fluid 950, such as blood. In the illustrated example, a conductive liquid medium 952 is in the lumen 944 and surrounding the electrode 920. In one example, the conductive liquid medium 952 includes blood that has seeped into the distal portion of the lumen 944. In another example, the conductive liquid medium 952 include saline that is passed into the lumen 944 as a saline flush from a proximal end of the delivery device 902. In other examples, the lumen 944 is filled with a non-conductive fluid. Examples of such a non-conductive fluid include dextrose and carbon dioxide.

FIG. 9B illustrates the assembly 901 and the crossing member 900 within the delivery device 902 after RF energy is applied to the electrode. An electrically insulative gaseous layer 930 forms around the heated electrode. In some puncture techniques, a clinician employs a running start strategy—in which the RF energy is applied to the electrode 920 within the delivery device 902 and retracted from the tissue and then, after the electrode is fully encapsulated in a bubble, the energized electrode 920 is physically advanced to contact and cross the target tissue. When subjected to heating from the applied RF energy, eventually bubbles on the electrode 920 will coalesce and encapsulate the electrode 920 with an electrically insulative gaseous layer 930 disposed between the conductive electrode 920 and the conductive liquid medium 952 within the lumen 944. The insulative gaseous layer 930 encapsulating the electrode 920 does not permit current in the electrode 920 from the applied RF energy to shunt into the conductive liquid medium 950, 952.

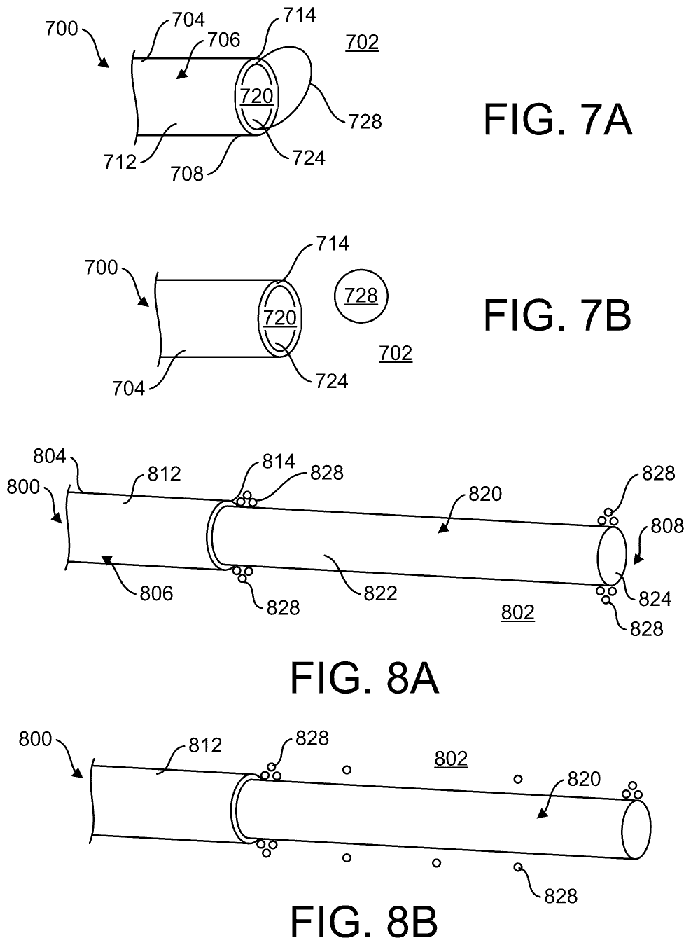

FIG. 10 illustrates the assembly 901 and crossing member 900 once the energized electrode 920 is extended from the distal end of the delivery device 902 toward target tissue 960. In the example, RF energy was applied to form an electrically insulative gaseous layer 930 while the electrode 920 was retracted within the lumen 944. After the electrode 920 is extended from the delivery device 902, the electrically insulative gaseous layer 930 remains on the electrode 920. As the electrode 920 is moved closer to the target tissue 960, the electrically insulative gaseous layer 930 is displaced by the tissue 960. In the illustrated example, the distal tip 908 of the crossing member 900 approaches the target tissue 960 and the gaseous layer 930 becomes thinner between the distal tip 908 and the target tissue as the insulative gaseous layer 930 is displaced. Eventually, the gap between the energized electrode 920 and the target tissue 960 reaches a distance such that an arc discharge occurs and causes a small vaporization of the target tissue as illustrated in FIG. 5B. The insulative gaseous layer 930 is thinned enough to cause a dielectric breakdown. In this embodiment, the target tissue 960 is vaporized before the electrode 920 directly contacts the target tissue 960.

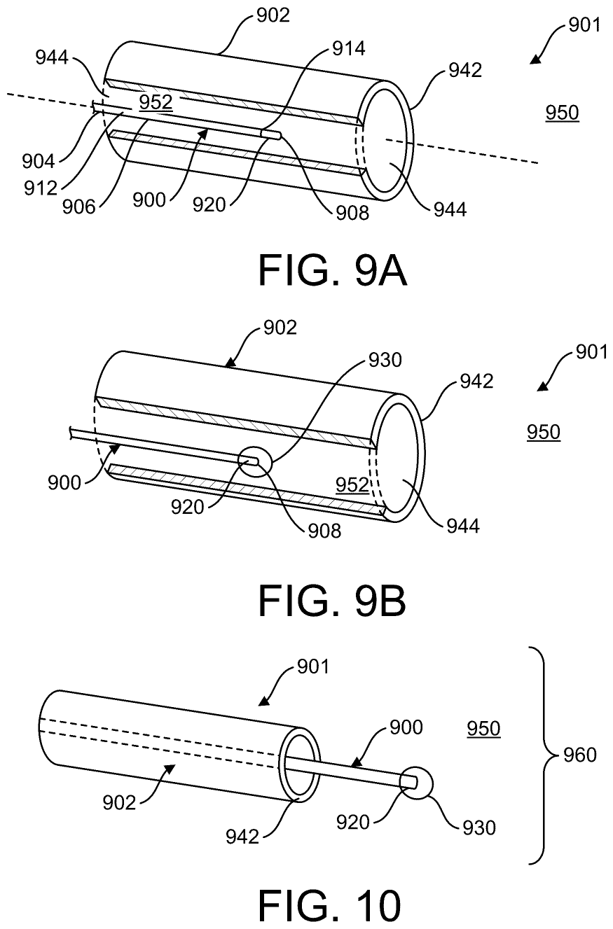

FIG. 11 illustrates an embodiment of electrosurgical unit, such as an electrosurgical generator 1100, which can correspond with electrosurgical generator 104 in system 100. The electrosurgical generator 1100 includes an RF output circuit 1102, a plurality of device connectors 1104 including an active connector 1106 and a return connector 1108, a measurement circuit 1110, a controller 1112. The generator 1100 can include other features such as output device such as a display or speakers and a user interface such as a touchscreen or keys (neither of which are shown) to receive user inputs and alert the user to conditions of the generator. In one embodiment, the RF output circuit 1102 is configured to generate an RF puncture signal at a frequency. The RF output circuit 1102 can include a power supply to provide a direct current signal and can convert the direct current signal to an alternating current signal. The RF output circuit 1102 is configured to generate a plurality of voltages, waveforms having various duty cycles, peak voltages, crest factors, frequencies and other suitable parameters and provide the selected RF puncture signal to the active connector 1106. The device connectors 1104 can be configured to include receptacles located on a housing of the RF generator 1100 that can be mechanically coupled to electrosurgical devices. The device connectors 1104 are configured to electrically couple the electrosurgical generator 1100 to various electrosurgical devices. For example, the active connector 1106 is suitable for electrically coupling the transeptal crossing device 154. The return connector 1108 is suitable for electrically coupling to the ground pad electrode 112 when an electrosurgical device is operated in a monopolar mode or to a return electrode on the electrosurgical device when operated in a bipolar mode. The measurement circuit 1110 is electrically coupled to the device connectors 1104 and is configured to determine current or voltage measurements from RF puncture signal generated by the RF output circuit 1102 and present the current and voltage measurements to the controller 1112. The measurement circuit 1110 can include circuit elements or paths electrically coupled to the RF output circuit 1102 or at least some of the output connectors 1104 including the active connector 1106 and return connector 1108 and is configured to provide a signal representative of the active and return voltages and active current. In one embodiment, the measurement circuit 1110 samples the RF puncture signal at a selected frequency. The circuit elements can include current probes to measure currents of interest. In one embodiment, the measurement circuit 1110 includes an analog to digital converter coupled to the circuit elements and the controller 1112 to provide digital signals to the controller 1112.

In embodiments, the controller 1112 is implemented with any combination of hardware and programming to configure the functions of electrosurgical generator 1100. In one embodiment, the programming for the electrosurgical generator 1120 include processor executable instructions stored on at least one non-transitory machine-readable storage medium, such as a memory device and the hardware includes at least one processing resource, such as a microprocessor, to execute those instructions. In other embodiments, the functionalities of electrosurgical generator 1100 and method may be at least partially implemented in the form of electronic circuitry. Examples of electronic circuitry include integrated circuits including ASICs and programmable logic devices, such as field programmable gate arrays.

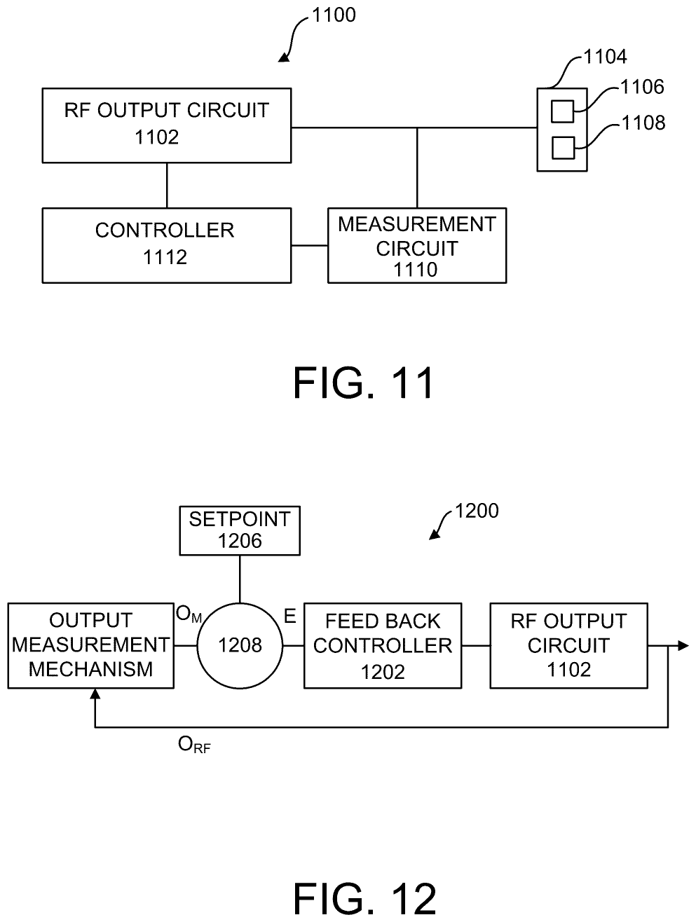

FIG. 12 illustrates an embodiment of a closed feedback loop controller 1200 implemented in the electrosurgical generator 1100 such as in controller 1112. In a closed feedback loop, the output of a system, such as the RF puncture signal output from the RF output circuit 1102, is fed back through measurement device, such as measurement circuit 1110, to digitize the output. The digitized output is processed, such as via controller 1112, to generate a feedback input for a comparison with a reference value to determine an error (difference between the feedback input and the reference value). A feedback controller 1200 takes the error to change the system under control, such as the RF output circuit 1102. The illustrated feedback loop controller 1200 includes a feedback controller 1202 and an output measurement mechanism 1204. The output measurement mechanism 1204 receives the output ORF of a system, such as an amplitude (voltage) or electrical current information of the RF puncture signal from the RF output circuit 1102, generates a mechanism output OM (or feedback input), such as a sampled, processed, waveform amplitude and phase data, and compares the mechanism output OM to a setpoint 1206 via comparator 1208 to generate an error E. The error E is provided to the feedback controller 1202, to change the output ORF under RF output circuit 1102 control. In embodiments, the setpoint 1206 is determined based on a user input or a configured input. In one example, the setpoint is selected from one or more available values based on one or more selectable RF puncture signals. The feedback controller 1202 and comparator 1208 can be implemented with a control loop feedback mechanism such as a proportional-integral-derivative (PID) mechanism and constructed in the controller 1112.

In this embodiment, voltage-feedback control can be more desirable than simple power-feedback control or simple current-feedback control. The impedance-independence of voltage feedback allows the selection of a consistent “feedforward” energy delivery setting at the start of therapy, regardless of the electric environment of the puncture electrode. By comparison, current-feedback and power-feedback control schemes implement more conservative initial settings to avoid large overshoot or undershoot for situations in which the impedance of the starting electrical load is outside of the nominal range. As impedance changes during the different phases of a puncture treatment, voltage feed-back control enables a reduced to minimal quantity of power to be delivered to the puncture electrode to achieve the task of tissue vaporization. Voltage-feedback configuration reduces the effects on hardware components such as power output of the generator, electrical insulation, conductor gauge, and dispersive ground pad. As the conductive electrode dynamically changes in size with the formation of the electrically insulative gaseous layer during tissue vaporization, the impedance of the electrode changes and the output current applied to the electrode decreases to maintain the selected current density. By controlling voltage, the overall power demands decrease during the progress of tissue vaporization.

In an alternative embodiment, the closed feedback loop controller 1200 implements a selective voltage-feedback control and a current-feedback control. For example, the output measurement mechanism can be configured to selectively measure and provide an output voltage or an output current to the comparator 1208. The setpoint 1206 can be switched between a voltage appropriate setpoint and a current appropriate setpoint, and the control loop feedback mechanism can be switched to respond to a voltage appropriate error and a current appropriate error.

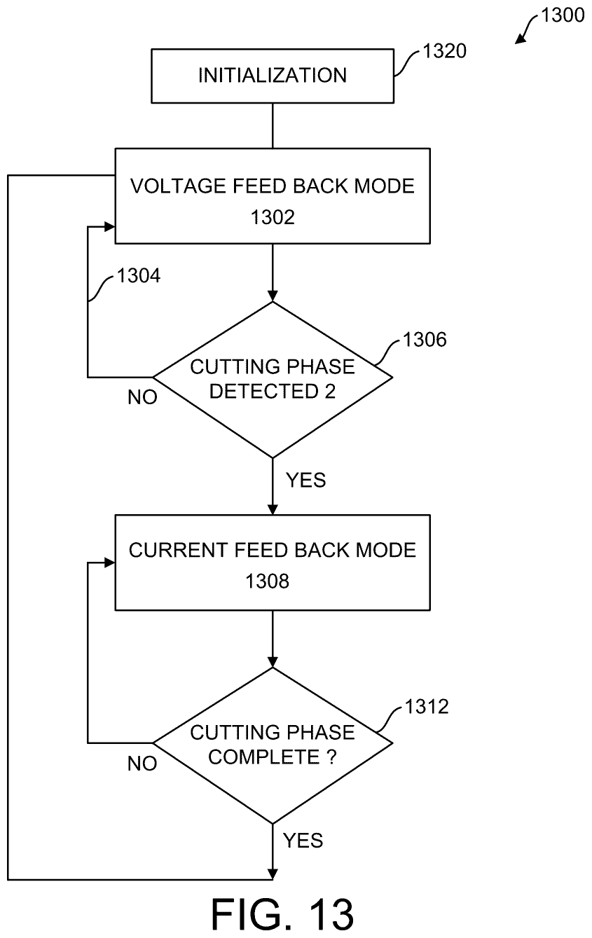

FIG. 13 illustrates an example process 1300 for configuration of the feedback loop controller 1200 in the alternative embodiment. Initially, the feedback loop controller is configured in a voltage-feedback control scheme as a voltage-feedback mode at 1302, such as after startup of the generator 104 but prior to the application of therapy at 1320. In voltage-feedback control mode at 1302, the measured output is a voltage and the setpoint corresponds with a selected voltage of the power delivery. The controller 1200 remains in voltage-feedback mode at 1304 until a cutting phase of the therapy delivery is selected or detected at 1306. Once the cutting phase is selected or detected 1306, such as when the clinician is prepared to vaporize tissue, the control scheme switches to a (electrical) current-feedback control mode at 1308. In current-feedback control mode at 1308, the measured output is an electrical current and the setpoint corresponds with a selected electrical current of the power delivery. The electrode can be energized and encapsulated with the gaseous electrically insulative layer. The control scheme remains in current-feedback mode 1308 during the entire duration of cutting phase of therapy delivery at 1310. Once the cutting phase of therapy delivery is complete at 1312, such as once the tissue is punctured or the system can detect a ramp up of impedance at the electrode as a result of a successful vaporization, the control scheme switches back to voltage-feedback mode at 1302.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An electrosurgical system configured to puncture biological target tissue, the electrosurgical system comprising:
an electrosurgical generator configured to generate a radiofrequency (RF) energy; and
a crossing member having a distal end adapted to be disposed within a conductive liquid medium proximate the biological target tissue, the distal end having an electrode adapted to deliver the RF energy,
wherein the electrode is configured to apply the RF energy to generate an electrically insulative gaseous layer within the conductive liquid medium to encapsulate the electrode and to vaporize the biological target tissue from within the electrically insulative gaseous layer.

2. The electrosurgical system of claim 1, wherein the electrosurgical generator is configured to drive a current and voltage within a selected range against a plurality of impedances.

3. The electrosurgical system of claim 1, wherein the electrode includes a surface area in a range between 1.2 square millimeters and 3.5 square millimeters and the electrosurgical unit provides 270 volts to the electrode.

4. The electrosurgical system of claim 1, wherein the electrode is an exposed section of an electrical conductor on the crossing member.

5. The electrosurgical system of claim 4, wherein the crossing member includes an electrically insulated shaft.

6. The electrosurgical system of claim 1, wherein the electrode includes a cylindrical shape.

7. The electrosurgical system of claim 6, wherein the electrode includes circular edges.

8. The electrosurgical system of claim 1, wherein the electrically insulative gaseous layer includes a coalesced bubble adhered to the electrode.

9. The electrosurgical system of claim 8, wherein the coalesced bubble is formed from a plurality of bubbles.

10. The electrosurgical system of claim 1, wherein the electrosurgical generator includes a feedback controller having a voltage-feedback control.

11. The electrosurgical system of claim 10, wherein the feedback controller further includes a selectable current-feedback control.

12. The electrosurgical system of claim 11, wherein the current-feedback control is selected during a cutting phase.

13. The electrosurgical system of claim 12, wherein the voltage-feedback control is selected when the cutting phase is complete.

14. The electrosurgical system of claim 1, wherein the crossing member is included in a crossing system having a sheath and a dilator.

15. The electrosurgical system of claim 1, wherein the crossing member is a transseptal guidewire.

16. A method of puncturing a biological target tissue with an electrode coupled to a source of radiofrequency (RF) energy, the method comprising:
positioning the electrode within a conductive liquid medium and proximate the biological target tissue;
while the electrode is within the conductive liquid medium, encapsulating the electrode with an electrically insulative gaseous layer;
vaporizing the biological target tissue via the RF energy applied to the electrode encapsulated by the electrically insulative gaseous layer.

17. The method of claim 16, wherein the electrically insulative gaseous layer includes coalesced bubbles adhered to the electrode.

18. The method of claim 16, wherein the electrode is disposed within a sheath, and the conductive liquid medium is disposed within the sheath.

19. An electrosurgical system configured to puncture biological target tissue, the electrosurgical system comprising:
an electrosurgical generator configured to generate a radiofrequency (RF) energy; and
a crossing member assembly having a delivery device defining a lumen and crossing member disposed within the lumen, the lumen configured to include a conductive liquid medium, the crossing member having a distal end adapted to be disposed within the conductive liquid medium, the distal end having an electrode adapted to deliver the RF energy,
wherein the electrode is configured to apply the RF energy to generate an electrically insulative gaseous layer within the lumen and the conductive liquid medium to encapsulate the electrode and to extend from the delivery device to vaporize the biological target tissue from within the electrically insulative gaseous layer.

20. The electrosurgical system of claim 19, wherein the delivery device is a sheath.
